(19) United States
(12) Patent Application Publication
(10) Pub. No.: US 2016/0102283 A1
(43) Pub. Date: Apr. 14, 2016

(54) MICROFLUIDIC DEVICE AND SYSTEM FOR PRECISELY CONTROLLING AND ANALYZING SHEAR FORCES IN BLAST-INDUCED TRAUMATIC BRAIN INJURIES

(71) Applicant: The United States of America as represented by the Secretary Department of Health and Human Services, Rockville, MD (US)

(72) Inventors: Rea Ravin, Rockville, MD (US); Nicole Morgan, Rockville, MD (US); Paul S. Blank, Rockville, MD (US); Joshua Zimmerberg, Rockville, MD (US)

(21) Appl. No.: 14/880,150

(22) Filed: Oct. 9, 2015

Related U.S. Application Data
(60) Provisional application No. 62/062,053, filed on Oct. 9, 2014.

(51) Int. Cl. C12M 1/42; C12M 1/34; C12M 1/00; C12M 3/06

(52) U.S. Cl. CPC C12M 35/04; C12M 23/16; C12M 41/38; C12M 23/38

(57) ABSTRACT

Embodiments of a microfluidic system and method for stimulating a blast shock wave that supports a short and defined laminar flow of a liquid media solution through a microfluidic device such that sheer stress on neural tissue disposed within the microfluidic device is precisely controlled are disclosed. The microfluidic system includes a pneumatic device applies a blast shock wave having a quick rise time across a microfluidic channel of the microfluidic device. The microfluidic device includes an inlet reservoir in fluid flow communication with an outlet reservoir through the microfluidic channel. The inlet and outlet reservoirs are secured to a top structure which is attached to a bottom structure that collectively defines the microfluidic channel with a cover slip that is attached to the underside of the bottom structure.

### CROSS REFERENCE TO RELATED APPLICATIONS

This is a non-provisional application that claims benefit to U.S. Provisional Application Ser. No. 62/062,053, filed on Oct. 9, 2014, in which the entire contents is herein incorporated by reference in its entirety.

### FIELD

The present disclosures relates to systems and methods for simulating and analyzing the effect of an explosion on biological tissue, such as tissue of the central nervous system (CNS), and in particular to a microfluidic device having a baffle to allow for precise control of shear forces with minimal pressure changes.

### BACKGROUND

Blast induced TBI (bTBI) remains an issue of great interest for the public, health and research communities. Since 2001, over 150,000 US military personnel have been diagnosed with mild TBI or concussion, often after exposure to an explosive blast, with a spectrum of neurological and psychological deficits. Due to difficulties in describing the origin of TBI, precise incidence statistics for mild bTBI are scarce. Understanding the mechanisms and pathology resulting from the primary injury phase of a blast, a direct result of the shockwave generated by an explosion, is still quite limited. The blast shock wave responsible for mild bTBI is a transient, solitary supersonic pressure wave with a rapid (sub-msec) increase in pressure (i.e. compression) followed by a more slowly developing (msec), below ambient pressure phase (i.e. tension). Although dynamic compression, tension, and shear stress have all been proposed to explain the deficits observed in mild bTBI, the identity of the mechanical forces involved, the tissue-force interaction(s) and the cellular damage properties remain unresolved. The brain is a complex system with compositional inhomogeneity, through which shock waves travel at different speeds. It is this difference in speed that potentially creates shear, between and within brain cells. The development of shear forces will depend on the orientation of the CNS tissues with the propagating shock wave. Thus, the different orientations of individuals or animals to a blast can result in different responses to the same blast.

Animal studies on the effects of shock wave in vivo fail to decouple the proposed direct effects of the pressure transient from the secondary effects of the shear stresses produced by that pressure transient. In vitro models of primary blast injury are likewise limited and don't differentiate shear from pressure. Therefore, it is critical to develop experimental methods simulating blast injury on human brain cells.

### BRIEF DESCRIPTION OF THE DRAWINGS

FIG. 1 is a simplified block diagram of a microfluidic system having a microfluidic device in operative communication with a pneumatic device for generating a simulation of a blast shockwave within the microfluidic device positioned on a microscope stage in which the effects of the blast shock wave are detected by an objective lens.

FIG. 2 is a perspective view of the microfluidic device connected to a plate through a holder.

FIG. 3A is a perspective view of the microfluidic device; FIG. 3B is a partial cross-sectional view of the microfluidic device; FIG. 3C is an enlarged view of the microfluidic device of FIG. 3B showing the baffle of the inlet reservoir of the microfluidic device; FIG. 3D is a side view of the microfluidic device; FIG. 3E is a top view of the microfluidic device; FIG. 3F is a cross-sectional view along line 3F-3F of the microfluidic device of FIG. 3E; and FIG. 3G is an end view of the microfluidic device.

FIG. 4A is a perspective view of the microfluidic device secured to a plate through a holder; FIG. 4B is an opposite perspective view of the microfluidic device of FIG. 4A; FIG. 4C is a partial cross-sectional side view of the microfluidic device secured to the plate through the holder; FIG. 4D is a partial cross-sectional end view of the microfluidic device secured to the plate through the holder; and FIG. 4E is a bottom view of the microfluidic device secured to the plate through the holder.

FIG. 5A is a side view of a pressurized gas tank for the pneumatic device; and FIG. 5B is an enlarged view of the pressurized gas tank of FIG. 5A.

FIG. 6A is a schematic of a first embodiment of the microfluidic channel having a serpentine configuration; FIG. 6B is a schematic of a second embodiment of the microfluidic channel having a multiple serpentine configuration; FIG. 6C is a third embodiment of the microfluidic channel having a 5 mm width; and FIG. 6D is a fourth embodiment of the microfluidic channel having a 10 mm width.

FIGS. 7A-7L show graphs depicting flow measurements of constant flow at a number of positions.

FIG. 8A is an first image of bead trajectories during a first few msec of a simulated blast shock wave; FIG. 8B is a second image of the same bead trajectories during the second part of the simulated blast shock wave; and FIG. 8C is a graph of the speed profiles of the bead trajectories over time during the simulated blast shock wave.

FIG. 9A is a graph showing the quadratic relationship between pneumatic tank pressure and velocity; and FIG. 9B is a graph showing the quadratic relationship between pneumatic tank pressure and shear stress.

FIG. 10 is a graph showing the pressure evaluation within the microfluidic channel.

FIG. 11A is a schematic diagram of the cover slip having a PDMS rectangular frame used to coat and plate cells to a restricted area; FIG. 11B is a schematic diagram of the cover slip having a PDMS well; and FIG. 11C is a schematic of the microfluidic device showing the microfluidic channel with a restricted area defined on the cover slip for defining and isolating a region for cellular growth in the microfluidic channel.

FIG. 12 is a graph showing a shear dependent calcium response of human dissociated CNS cell cultures.

Corresponding reference characters indicate corresponding elements among the view of the drawings. The headings used in the figures do not limit the scope of the claims.

### DETAILED DESCRIPTION

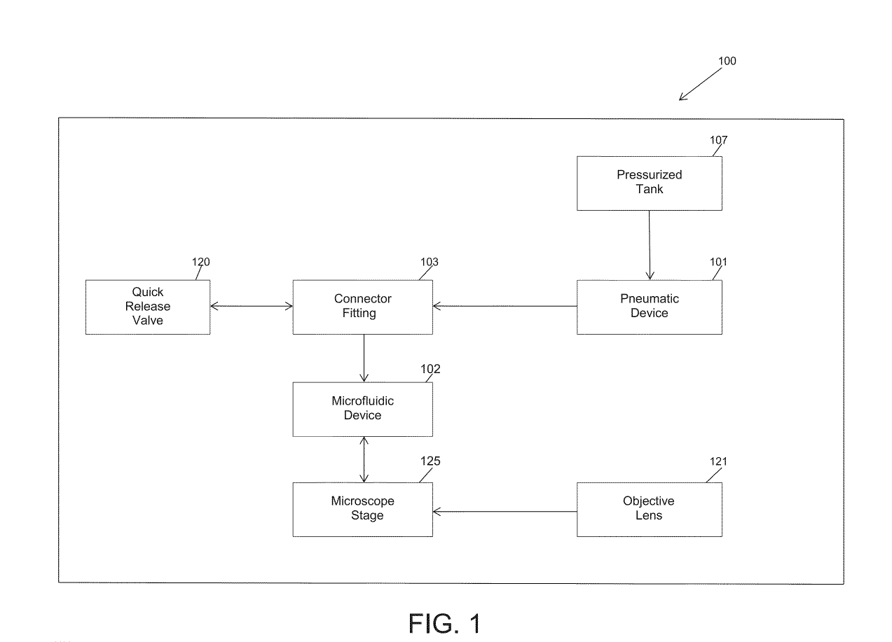

This disclosure generally relates to systems, devices, and methods for simulating precise shear forces leading to brain injury in response to a blast shock wave. The disclosure further relates microfluidic systems, devices, components, and methods for manufacturing, assembling, and using a microfluidic device to observe the effects of a blast shock wave that produces a short and defined laminar flow through a liquid media solution creates defined shear forces in the microfluidic device. Referring to the drawings, embodiments of a microfluidic system are generally indicated as 100 in FIGS. 1-12.

A microfluidic system 100 has been developed that mimics the temporal properties of a simulated blast shock wave and is integrated within a state-of-the-art optical microscope allowing for direct observation of the effect of a blast on human brain cells. As illustrated in FIG. 1, one embodiment of the microfluidic system 100 provides a means for simulating precise shear forces leading to brain injury within the setting of a microfluidic device 102 in response to a simulated blast shock wave to control the shear forces and support both laminar flow within the microfluidic device 102 over the time scales of the blast shock wave with minimal pressure changes. In some embodiments, the microfluidic device 102 is in communication with a pneumatic device 101 that initiates a blast shock wave through a connector fitting 103 that is introduced to the microfluidic device 102.

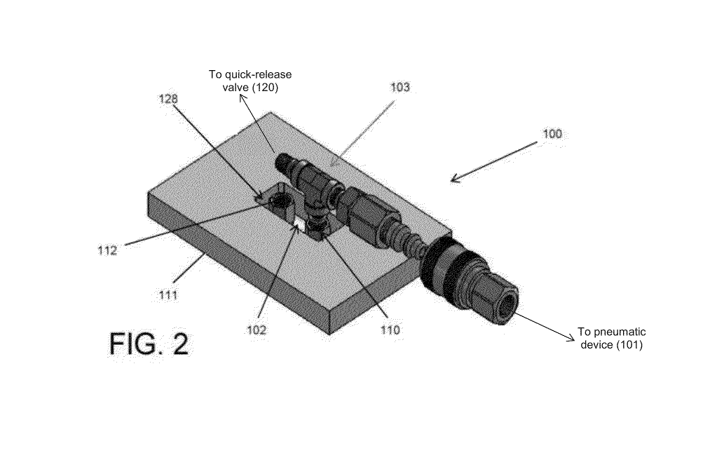

Referring to FIG. 1, the pneumatic device 101 is in communication with a pressurized gas tank 107 that provides a pressurized gas to the pneumatic device 101 for initiating a blast shock wave within the microfluidic device 102. In some embodiments, the pneumatic device 101 is in fluid flow communication with a quick release valve 120 positioned on an opposite side of a connector fitting 103, such as a T-connector, that allows pressure to build up within the connector fitting 103 during the blast shock wave to a predetermined level. Once pressure inside the connector fitting 103 exceeds this predetermined pressure level, the quick-release valve 120, such as a plug, is ejected from the quick-release valve 120 to release the pressurized gas from the connector fitting 103 and terminate the blast shock wave. In other embodiments, the quick-release valve 120 may be a fixed valve that releases the pressurized gas from the connector fitting 103 after the pressure reaches a predetermined level. In this arrangement, the blast shock wave produces a quick rise in pressure within the connector fitting 103 followed by a drop in pressure as the pressure of the blast shock wave is released by the quick-release valve 120, thereby terminating the blast shock wave initiated by the pneumatic device 101. As the pressure builds up inside the connector fitting 103, a liquid solution that completely fills the microfluidic device 102 attached to the connector fitting 103 is forced through the microfluidic device 102 in a short and defined laminar flow from the inlet to the outlet of the microfluidic device 102. As shown in FIG. 2, in some embodiments, the connector fitting 103 is configured to provide three-way fluid flow communication between microfluidic device 102, the pneumatic device 101 and the quick-release valve 120. In some embodiments, the microfluidic device 102 is positioned on a microscope stage 125 such that the human brain cells in culture within the microfluidic device 102 may be imaged by an objective lens 121 for obtaining a series of images of the cells during exposure to short and defined laminar flow initiated by the blast shock wave within the connector fitting 103.

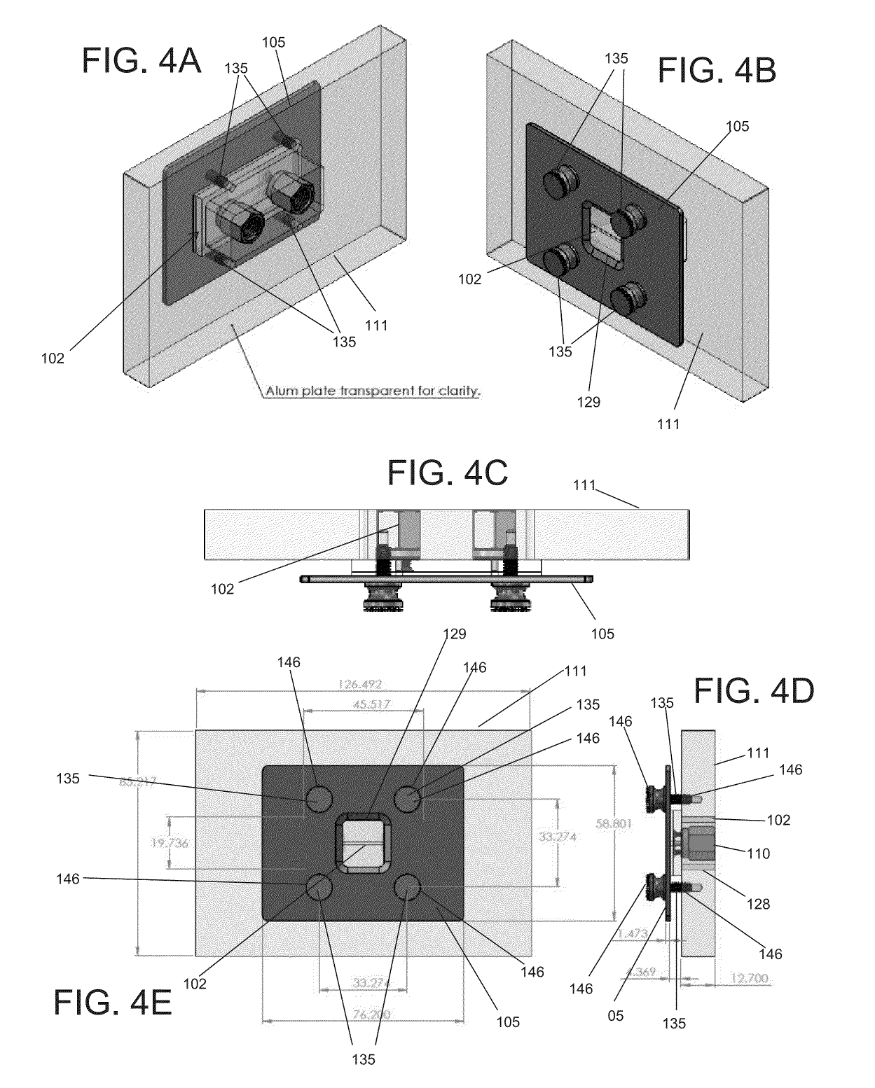

As further shown in FIGS. 2 and 4A-4E, the microfluidic system 100 further includes a holder 105 for securing the microfluidic device 102 to a plate 111, such as an aluminum plate, when assembling the microfluidic system 100. In one arrangement, the microfluidic device 102 is secured within a plate opening 128 (FIG. 4D) defined by the plate 111 through the holder 105. The holder 105 and the plate 111 collectively define a plurality of aligned apertures 146 (FIG. 4E) formed through the plate 111 and holder 105, which are configured to receive a respective securing member 135 for securing the holder 105 to the plate 111, thereby securely engaging the microfluidic device 102 to the plate 111. In some embodiments, the holder 105 defines a central opening 129 that provides access for imaging the cells in the microfluidic channel 108 of the microfluidic device 102 when engaged to the plate 111.

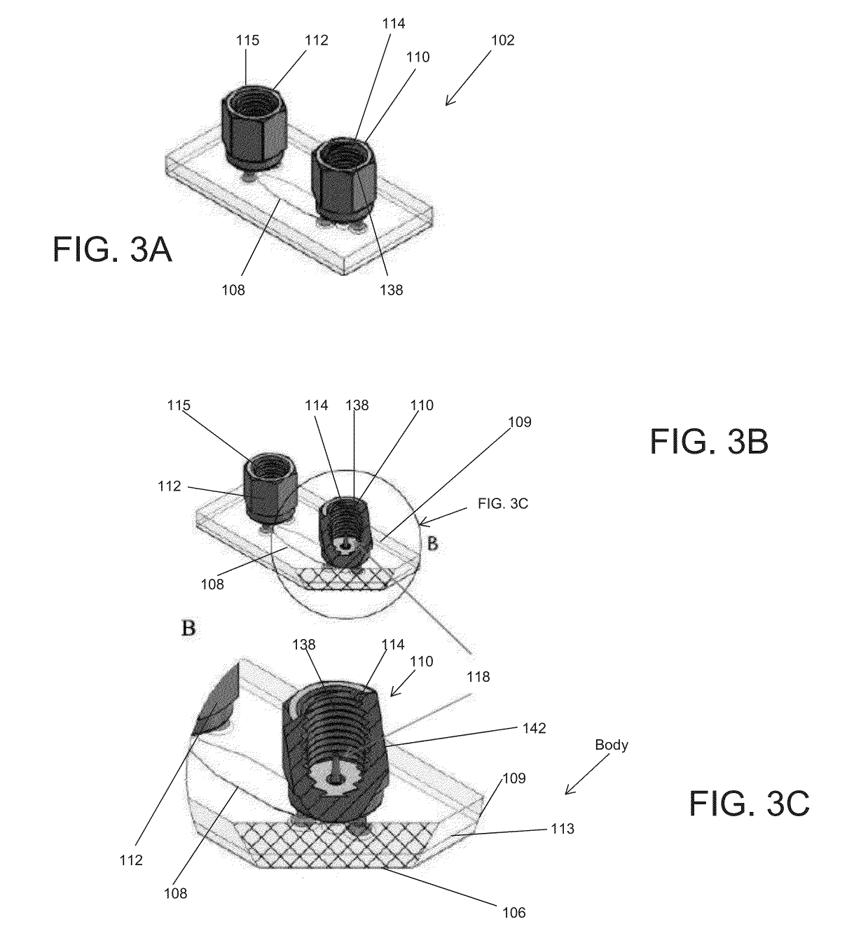

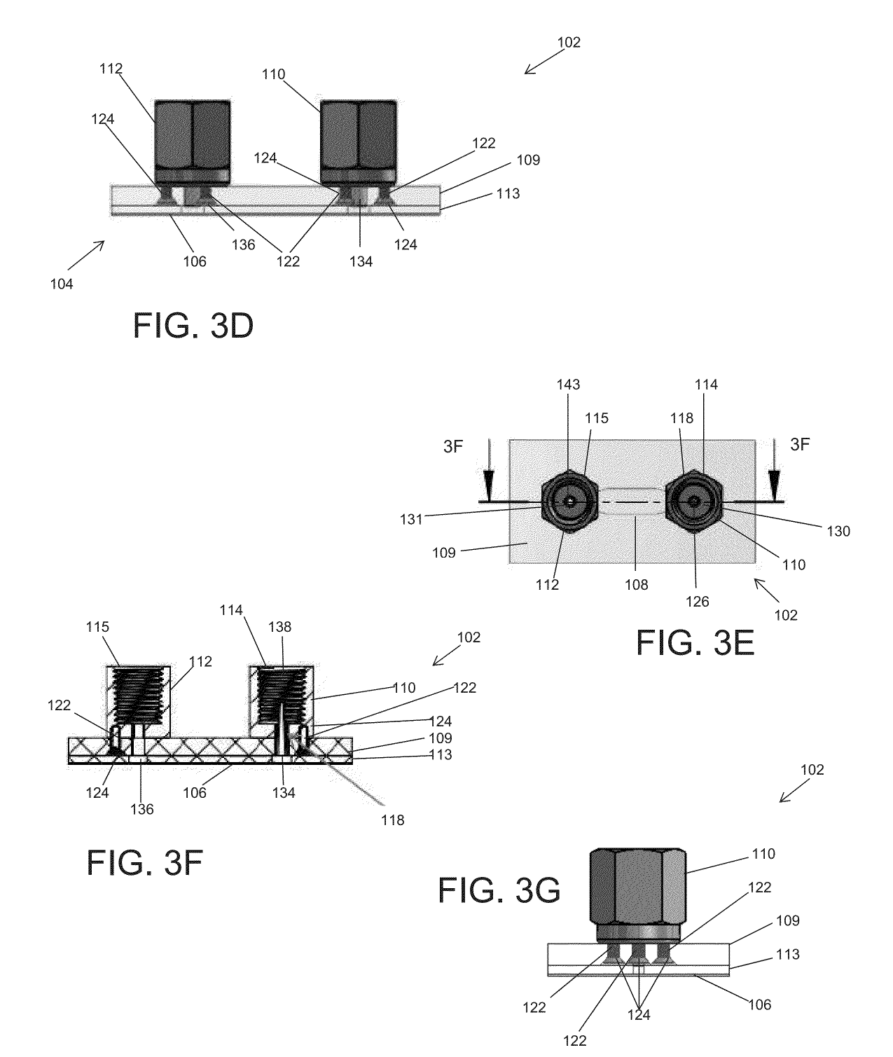

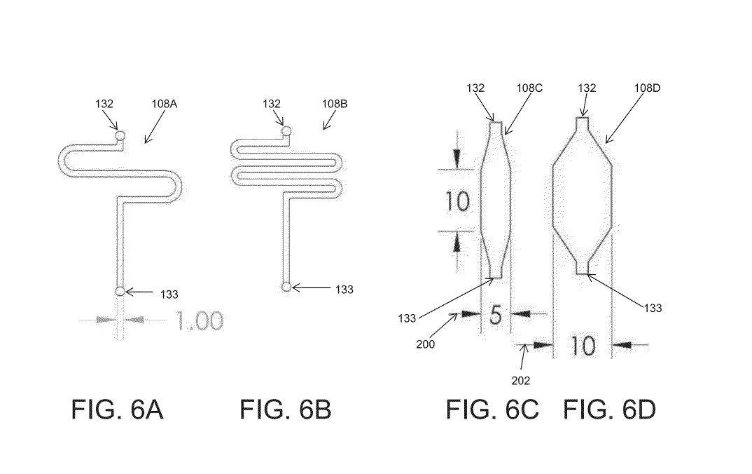

Referring to FIGS. 3A-3G, in some embodiments the microfluidic device 102 includes a top structure 109 secured to an inlet reservoir 110 and an outlet reservoir 112 in fluid flow communication with each other through a microfluidic channel 108 defined by the bottom structure 113, which is attached to the underside of the top structure 109. In some embodiments, the inlet reservoir 110 communicates with an inlet 132 (FIGS. 6A-6D) of the microfluidic channel 108 through an inlet conduit 134 that extends between the inlet reservoir 110 and the microfluidic channel 108. Similarly, the outlet reservoir 112 communicates with an outlet 133 (FIGS. 6A-6D) of the microfluidic channel 108 through an outlet conduit 136 that extends between the outlet reservoir 112 and the microfluidic channel 108. As shown in FIGS. 3D and 3F, the inlet and outlet conduits 134 and 136 extend through the top structure 109 from the inlet and outlet reservoirs 110 and 112, respectively, and communicate with opposite ends of the microfluidic channel 108 defined by the bottom structure 113 formed adjacent and directly underneath the top structure 109. In some embodiments, the top structure 109 may be a slide structure made from a plastic material and the bottom structure 113 may be made from polydimethylsiloxane (PDMS). In some embodiments, a cover slip 106 is attached adjacent and directly underneath the bottom structure 113 to collectively form the bottom surface of the microfluidic channel 108 with the bottom structure 113.

Referring to FIGS. 3A-3F, the inlet reservoir 110 and outlet reservoir 112 have substantially the same structural features. In particular, the inlet reservoir 110 defines an inlet opening 130 that communicates with an inlet chamber 114 of the inlet reservoir 110 configured to receive a liquid solution therein. Similarly, the outlet reservoir 112 defines an outlet opening 131 that communicates with an outlet chamber 115 of the outlet reservoir 112 configured to allow fluid to exit the microfluidic device 102. As further shown, the inlet reservoir 110 defines first internal threads 138 configured to engage the connector fitting 103 according to one arrangement of the microfluidic system 100. As shown in FIGS. 3D and 3F, in some embodiments a plurality of apertures 122 are defined through the top structure 109 which are configured to receive a respective securing member 124, such as a screw, to secure the inlet and outlet reservoirs 110 and 112 to the top structure 109 when assembling the microfluidic device 102. In some embodiments, three securing members 124 may be used to secure the inlet and outlet reservoirs 110 and 112, respectively, to the top structure 109 through respective apertures 122 defined through the top structure 109 as shown in FIG. 3G.

In addition, the inlet reservoir 110 includes a baffle 118 to produce uniform flow through the microfluidic channel 108 during a blast shock wave. In particular, the baffle 118 provides a barrier for preventing turbulent and vibratory flow from entering the microfluidic channel 108 when the inlet reservoir 118 is operatively connected to the pneumatic device 101 through the connector fitting 103 as shown in FIG. 2. Referring to FIGS. 3C and 3E, in some embodiments the baffle 118 extends outwardly from the bottom of the inlet chamber 114 and defines a baffle opening 142 having a restricted or extremely small aperture that communicates with the inlet chamber 114 at one end and the inlet conduit 134 at an opposite end of the baffle 118. In addition, the outlet reservoir 112 defines an outlet opening 143 (FIG. 3E) formed through the bottom surface of the outlet reservoir 112 that communicates with the outlet chamber 115 at one end and the outlet conduit 136 at an opposite end thereof such that fluid flow communication is established between the inlet reservoir 110 and the outlet reservoir 112 through the microfluidic channel 108.

The structural arrangement of the baffle 118 within the inlet reservoir 110 requires the liquid solution within the inlet chamber 114 to be forced by a short pressure pulse in milliseconds applied by the build-up and termination of the blast shock wave generated within the connector fitting 103 to flow through the restricted aperture of the baffle opening 142 before entering the microfluidic channel 108. As such, the baffle 118 supports a short and defined laminar flow of the liquid solution through the microfluidic channel 108 over the time scales of the blast shock wave as the short and defined laminar flow of the liquid solution travels from the inlet reservoir 110 and through the microfluidic device 102 before exiting from the outlet reservoir 112.

In one method of operation, the inlet reservoir 110 and the outlet reservoir 112 are filled completely with a liquid solution, such as a cell culture media, so that the liquid solution fills the entire microfluidic channel 108 and reaches an equal level in both the inlet and outlet reservoirs 110 and 112. A blast shock wave is then initiated by the operation of the pneumatic device 101 that quickly raises the level of pressure within the connector fitting 103. Once the pressure within the inlet reservoir 110 reaches a predetermined level, the quick-release valve 120 is actuated that releases the pressure within the connector fitting 103, thereby terminating the blast shock wave and producing a short and fast movement of the liquid solution within the microfluidic device 102. The short and fast movement of the liquid solution through the baffle 118 in the millisecond range generates a short and defined laminar flow of the liquid solution that travels through the microfluidic channel 108 before exiting the microfluidic device 102 at the outlet reservoir 112. In this manner, the shear forces through the microfluidic channel 108 may be precisely controlled.

In one embodiment, the microfluidic channel 108C (FIG. 6C) may have a height of 100 μm and a width 200 of 5 mm in which the width of 5 mm was found to support laminar flow, while in another embodiment of the microfluidic channel 108D (FIG. 6D) having a height of 100 μm and a width 202 of 10 mm was found not to support laminar flow. In one embodiment, the microfluidic channel 108A may define a single serpentine fluid pathway between an inlet 132 and an outlet 133 (FIG. 6A), while in other embodiments the microfluidic channel 108B may have a plurality of serpentine fluid pathways defined between the inlet 132 and the outlet 133 (FIG. 6B). In other embodiments, the microfluidic channels 108C and 108D each define substantially straight fluid pathways between the inlet 132 and outlet 133 (FIGS. 6C and 6D). In some embodiments, the microfluidic channel 108 may have a symmetrical configuration, an asymmetrical configuration, a rectangular configuration, a tapered configuration, and/or rounded configuration.

During experimentation, the microfluidic system 100 was found capable of generating reproducibly delivered blast shock waves with shear and minimal pressure changes to the same cells at different points of time and to follow their responses for long (24 hours) periods of time. In the previous work disclosed in related U.S. patent application Ser. No. 13/748,410, which has been incorporated by reference, it was demonstrated that human brain cells in culture are indifferent to blast induced transient pressure waves known to cause mild bTBI. However, when sufficient shear forces are present with shockwave pressure, calcium waves propagate throughout the cellular network of human central nervous system (CNS) dissociated cultures. The cell survival was unaffected 20 hours after shockwave exposure. These results suggest that shear forces have a role in how blast shock wave exposure leads to mild bTBI in brain cells in vivo and call attention to the need to characterize the response of CNS cells to shear in the absence of pressure transients. The influence of a controlled shear stress on cells in general and neurons in particular has been investigated in a variety of model systems including a rotating cone, linear actuator, and micro-fluidic-vacuum transfection. This work indicated that the rise time of an insult shear force is an important parameter in which faster rise times have larger cellular effects. The shear stress produced in these models have relatively slow rise time of 20 msec and slower. Models for the shear forces in a human brain as a result of a blast shock wave predict sub or msec rise time. Cells survive shear stress up to 14 Pa with 20 msec and longer rise times, but in neuronal culture cell membrane permeability to soluble dyes and electrical activity is altered. The prior art microfluidic systems may precisely control the pressure profiles, but have limited control of shear forces. Towards the goal of better understanding bTBI, in particular, the primary injury phase associated with mild bTBI, the microfluidic system 100 has been developed that allows precise control of shear forces with minimal pressure changes. In particular, the microfluidic system 100 allows precise control of the shear forces over a time interval and kinetics associated with a blast shock wave (sub msec rise times and msec durations).

The microfluidic device 102 was found to control shear forces and support both laminar flow over the time scales of blast shock waves with minimal pressure changes. In microfluidic channels with lateral dimensions below a few millimeters, the flow of incompressible fluids such as water and culture media at typical flow rates is generally laminar rather than turbulent. The transition between laminar and turbulent flow is determined by the Reynolds number, given by Re=p*v*DH/μ, where p is the fluid density, v is the average fluid velocity, μ is the dynamic viscosity, and DH=2*w*h/(w+h) is the hydraulic diameter of the microfluidic channel. For microfluidic channels with smooth walls, flow is fully laminar provided the Reynolds number is below 2300. The microfluidic channel 108 has some effective wall roughness because of the protrusion of the cell bodies into the microfluidic channels. For the channel heights of microfluidic channels 108 discussed herein, the resulting roughness corresponds to <10% of the height of the microfluidic channel 108. Laminar flow is still expected to persist at Reynolds numbers below 2300.

In laminar flow, the flow streams within the microfluidic channel 108 are parallel, with velocity values that vary in a well-characterized manner as a function of the type of flow and the distance from the channel wall. For pressure-driven flow, the velocity distribution is parabolic, with maximum fluid velocity in the middle of the microfluidic channel 108 and maximum shear forces at the microfluidic channel 108 walls, where the no-slip boundary condition is enforced. For the shallow and wide microfluidic channels 108 considered here, this means that cells on the bottom wall of the microfluidic channel 108 will experience relatively uniform and well-characterized shear forces as a function of volume flow rates, provided they are at least a distance comparable to the microfluidic channel 108 height from the side walls, and independent of their position along the microfluidic channel 108, provided they are not in the entrance region (e.g., inlet 132 and outlet 133) of the microfluidic channel 108. The microfluidic system 100 can assist in better understanding how the physical forces (shear and pressure) alter the short and long-term response of the CNS.

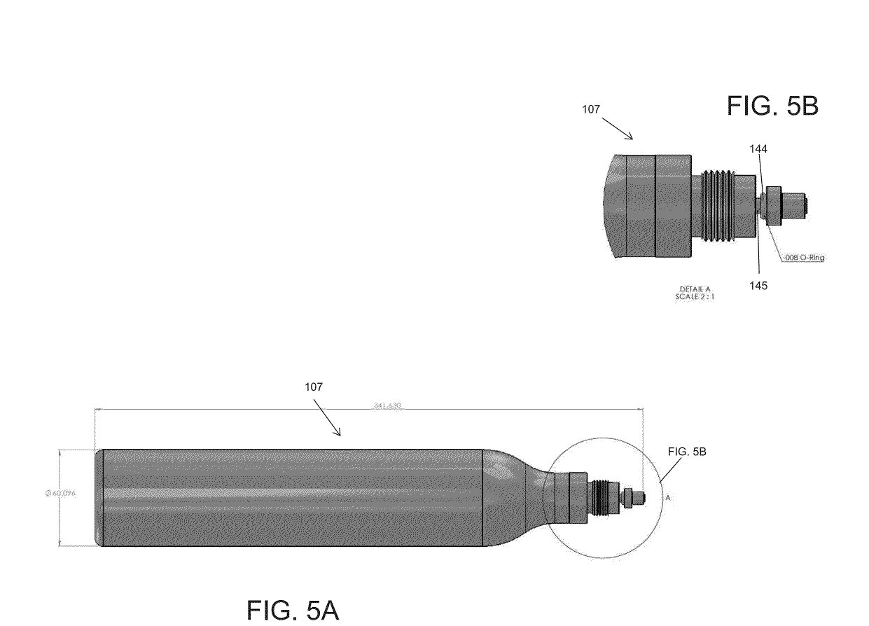

The microfluidic device 102 allows for precise control of shear to control fluid flow with minimal pressure transients associated with the pressure shock wave. The microfluidic device 102 is compatible with high resolution, time-lapse, optical microscopy using the objective lens 121. In the experiment, an existing pneumatic device 101 was modified to work with the microfluidic device 102 by reducing the amount of gas released in each shock wave exposure (blast) from a pressurized gas tank 107 shown in FIGS. 5A and 5B. This arrangement was implemented by: 1) reducing the tank pressure of the pressurized gas tank 107 from 1,500 PSI to 500 PSI, 2) reducing the size of the valve 145 on the pressurized gas tank 107, 3) restricting the travel distance of the valve 145 by adding 0-rings 144 between the valve 145 and the pressurized gas tank 107; 4) replacing the spring mechanism (not shown) of the pneumatic device 101 with a weaker spring (not shown); and 5) reducing the hammer weight of the pneumatic device 101.

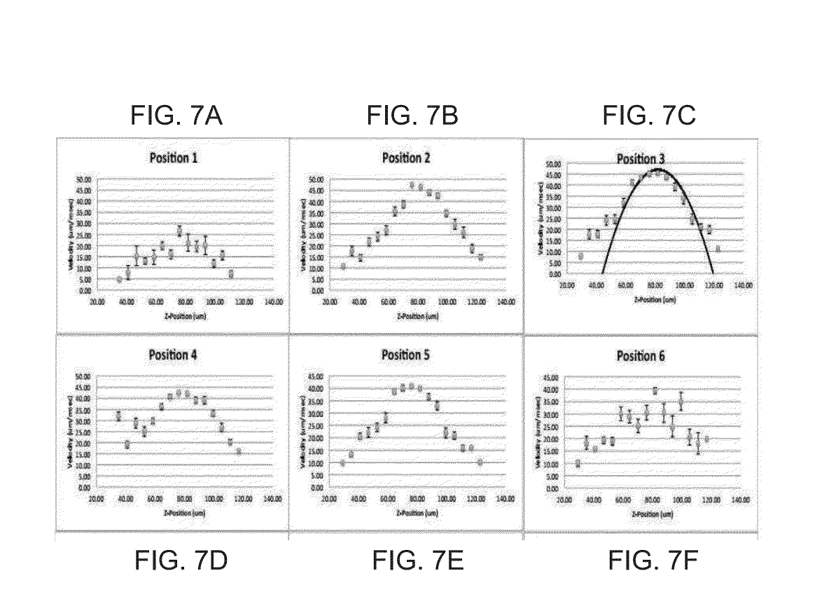

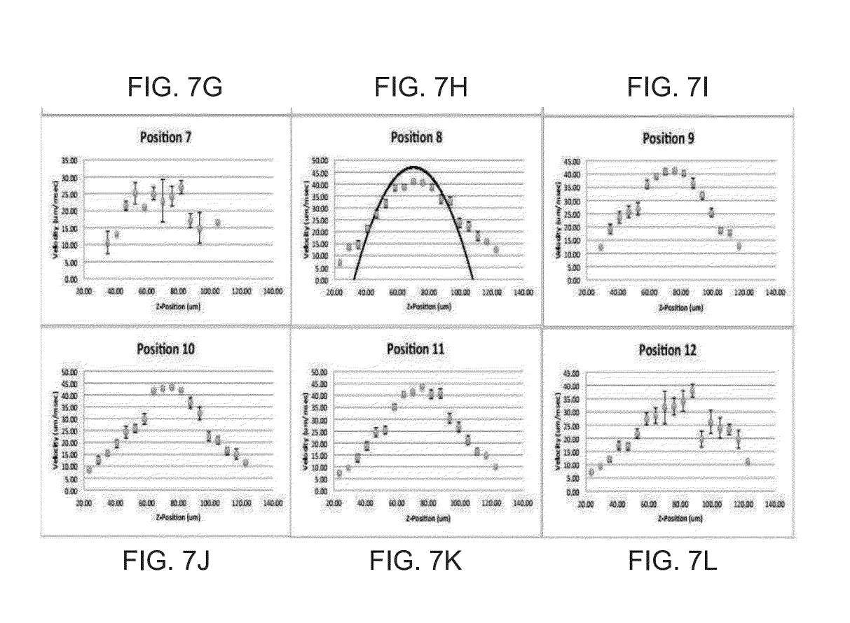

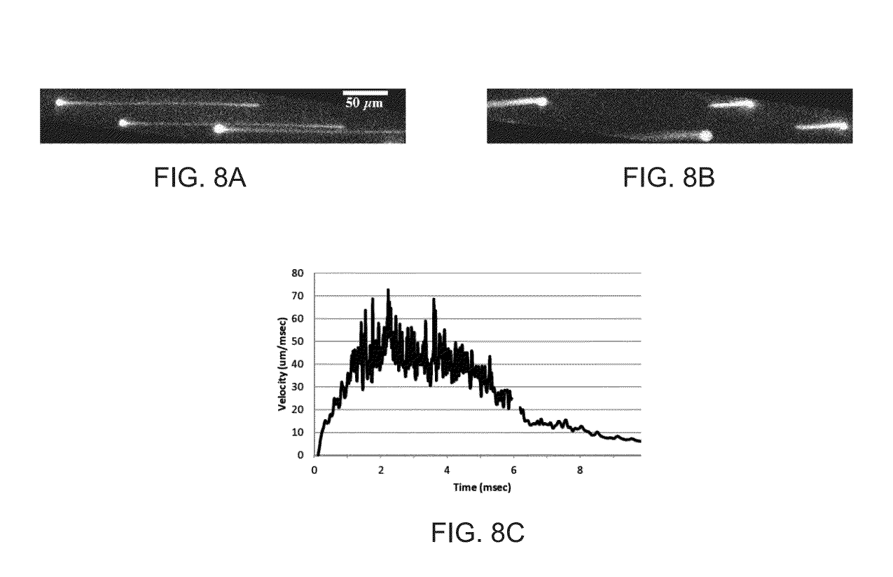

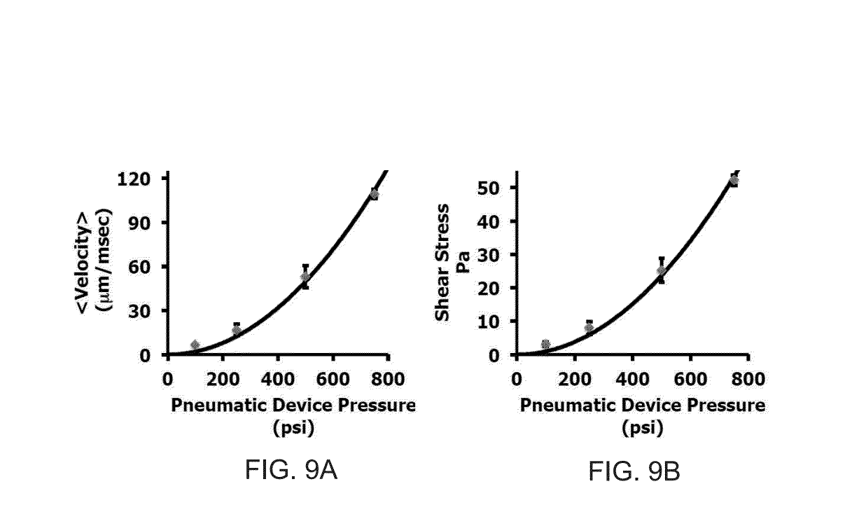

As shown in FIGS. 6A-6D, in some embodiments the height of the microfluidic channels 108A/108B is set at 100 um to preserve laminar flow using similar volume flow rates previously tested. Optimal flow properties were obtained using the microfluidic-channel 108C shown in FIG. 6C. Using this microfluidic channel 108C, constant volume flow was measured using fluorescent beads imaged with fluorescence microscopy. The flow fields measured in 12 different locations in the microfluidic channel 108C demonstrated laminar flow. Two sets of 6 locations, separated by 5 mm and spanning the width of the microfluidic channel 108C were collected. The six locations in each set were separated by 1 mm. The measured profiles were compared to the theoretically expected flow calculated from the maximal flow observed in the center of the microfluidic channel 108C as shown in FIGS. 7A-7L with good agreement was observed in the center of the microfluidic channel 108C (FIGS. 7C and 7H). As expected, on the wall side of the microfluidic channel 108C (FIGS. 7A, 7F, 7G and 7L) the flow was slower because of the effect of the walls of the microfluidic channel 108C. The parameters of the flow field during blast were measured in the 5 mm wide channel. It was observed that different beads have similar linear trajectories and no evidence for beads moving in and out of the focal plane; observations consistent with laminar flow during the blast (FIGS. 8A and 8B). The bead trajectories during blast are characterized by sub-msec rise times, a period of approximately constant flow (plateau), and decay to zero flow beginning at ˜5 msec (FIG. 8C). The average plateau speed was calculated by converting segment intensity into segment time using the intensity fraction (segment intensity/total trajectory intensity) multiplied by the exposure time. The segment length was then divided by segment time to give an average speed during the plateau. Different microfluidics channel flow rates were achieved by using different tank pressures (100-750 psi) in the pneumatic device 101. The relationship between pneumatic tank pressure and channel flow velocity at the coverslip was quadratic (FIG. 9A).

Maximum shear stresses, τ, were calculated using channel flow velocity and the physical properties of the channel as described in the methods. The conversion of velocity to shear stress, as a function of different pressurized gas tank 107 pressures, is shown in FIG. 9B and allows us to estimate the shear stress that cells experience using different tank pressures.

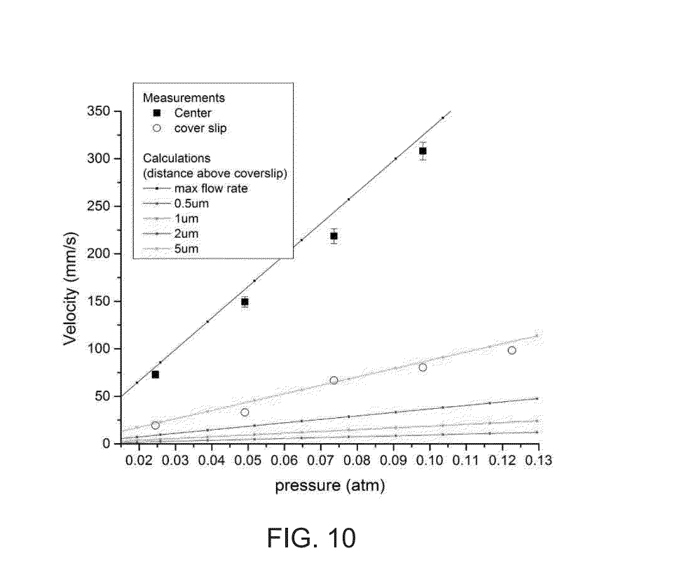

To determine the pressures in the microfluidics chamber during blast experiments, bead velocities were calibrated using known pressures. The trajectories of 2 μm fluorescent beads diluted 1/5000 in PBS were measured under constant flow conditions by varying the heights between the inlet reservoir 110 and the microfluidics chamber (different pressure drops). Beads were imaged at the cover slip and in the center of the channel to record both the slowest and fastest trajectories. Beads trajectories were measured as a function of hydrostatic pressure (height of the fluid reservoir above the microfluidics chamber). Maximal bead trajectories corresponding to velocities of ˜100 μm/msec were achieved with a pressure difference across the channel of less than 0.13 atm (FIG. 10). Theoretical calculations for the dependence of flow velocity on channel pressure at different positions above the boundary bottom indicate that our measurements at the coverslip corresponded to flow sampled ˜5 μm above the cover slip surface (FIG. 10).

In order to obtain a uniform flow during the blast the baffle 118 was added to the inlet reservoir 110 which is connected to the pneumatic device 101 (FIG. 2). It was discovered that the baffle 118 provides a barrier for turbulent and vibrating flow entering the microfluidics channel 108. Referring to the graph shown in FIG. 8C, the bead trajectories during blast with the baffle 118 are characterized by sub-msec rise times with a total duration −6 msec.

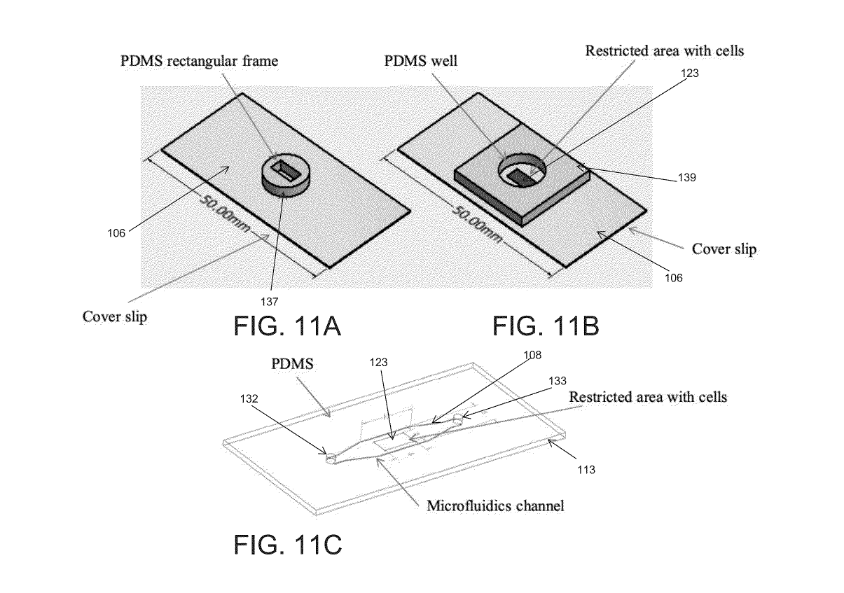

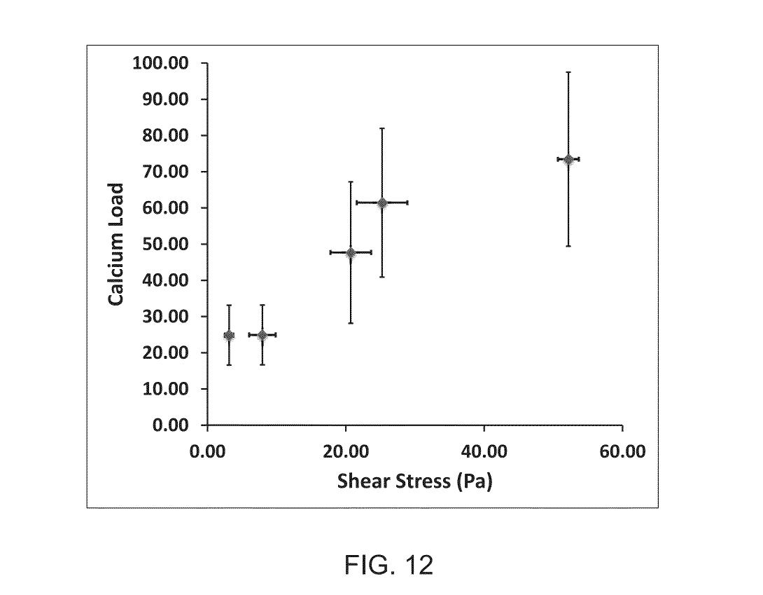

In order to confine the cell growth to specific restricted areas on a cover slip 106 which forms the bottom portion of the microfluidic device 102, a method was developed to coat a cell plating region 123 of the cover slip 106 with extra cellular matrix and later to grow the cells on that specific area without leaving any material residue on the rest of the cover slip 106. Any residue between the cover slip 106 and the bottom structure 113 can prevent the seal between the components of the microfluidic device 102. In some embodiments as shown in FIG. 11A, a PDMS frame 137 defines a rectangular 3×6 mm PDMS well. The PDMS frame 137 was attached to the center of 50×24 mm cover slip 106. The corner of the 3×6 mm PDMS well is marked on the bottom side of the cover slip 106 to locate the area with cells. The cover slip 106 inside the rectangular PDMS well was coated with extra cellular matrix, Primary, human CNS dissociated cells were plated inside the rectangular well. The next day the PDMS frame 137 was removed and a PDMS well 139 that creates a 10 mm diameter well was placed over the cells as illustrated in FIG. 11B (e.g., the bottom of the frame well is the cover slip 106 with the cells). Neural basal (NB) media supplemented with B27 was added to fill the wells; half of the media volume was changed twice a week, Cells are cultured for 2 to 3 weeks before use. Prior to experimentation, cells were labeled with the calcium indicator Fluo-4 AM. Using the previously marked corner of the 3×6 mm hole the microfluidic device 102 is assembled in such a way that the cell plating region 123 is located at the bottom center of the microfluidic channel 108 as shown in FIG. 11C. The microfluidic device 102 is assembled in a manner that does not alter cell survival, and maintains the cells for the length of the experiment. The inlet and outlet reservoirs 110 and 112 are filled with NB media supplemented B27, or any other type of media solution. A gentle suction is applied to the outlet reservoir 112 to completely fill the microfluidic device 102 with the media solution. The connector fitting 103 is connected to the inlet reservoir 110, thereby operatively connecting the pneumatic device 101 to the microfluidic device 102. Baseline images were collected every second for 100 seconds. A blast shock wave was triggered after the 100th image while continuously imaging the microfluidic device 102 for 10 minutes. Calcium signaling was evaluated over the entire field of view by averaging fluorescence, ΔF/F, as a function of time and then integrating the area under the ΔF/F curve for each experiment. The measurements indicate that the pressures developed within the microfluidics channel are low; for example, a pressure drop of 0.07 atm is created during a flow of 50 μm/msec, and the shear stress associated with this flow is sufficient to initiate a calcium response in CNS cells. The relationship between shear stress and the calcium response of human dissociate CNS cells is shown in FIG. 12. At low shear stress, <8 Pa, there is lower calcium response. At shear stress levels above 8 Pa there is a robust calcium response, suggesting that the shear stress threshold is between 8-25 Pa.

These current results demonstrate a calcium response threshold at the range of 20 pa in response to shear forces driven by laminar flow with minimal pressure transient. The threshold for calcium response in our previous experiments was around 1 pa, in these experiments the shear forces were the results of flow that was not laminar and was accompanied with pressure transient. This difference in threshold can result of: a) estimation only included the 2-D values that were quantifiable and did not take into account heterogeneity, b) shear may have developed as a result of turbulence and consequently, not well defined compared to laminar flow, and c) there may exist a synergism between pressure and shear and reduce the shear required for a response.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

1. A microfluidic system for precisely controlling shear forces generated by a blast shock wave, the system comprising:
a microfluidic device comprising:
a top structure attached to a bottom structure in which the bottom structure defines a microfluidic channel having a first end and a second end, the microfluidic channel including an inlet at the first end and an outlet at the second end;
an inlet reservoir engaged to the top structure and in fluid communication with the inlet of the microfluidic channel, wherein the inlet reservoir includes a baffle defining a restricted opening; and
an outlet reservoir engaged to the top structure and in fluid communication with the outlet of the microfluidic channel;
a liquid media solution that fills the microfluidic device; and
a pneumatic device in fluid communication with the microfluidic device to deliver a pressurized gas that generates a blast shock wave having a quick rise and fall in pressure that causes a short and fast movement of the liquid media solution through the restricted opening of the baffle and supports a laminar flow of the liquid media solution through the microfluidic channel.

2. The microfluidic system of claim 1, wherein the baffle extends within the inlet reservoir and communicates with the inlet of the microfluidic channel.

3. The microfluidic system of claim 1, further comprising:
an inlet conduit in fluid communication between the baffle and the inlet of the microfluidic channel; and
an outlet conduit in fluid communication between the outlet reservoir and the outlet of the microfluidic channel.

4. The microfluidic system of claim 1, further comprising:
a cover slip attached to the bottom structure, the cover slip forming a bottom surface of the microfluidic channel.

5. The microfluidic system of claim 1, further comprising:
a holder secured to the microfluidic device, the holder defining a plurality of apertures configured for receiving a respective securing member for securing the holder to the microfluidic device.

6. The microfluidic system of claim 1, wherein the microfluidic channel has a height of approximately 100 μm.

7. The microfluidic system of claim 1, further comprising:
a connector fitting in fluid communication with the inlet reservoir, the connector fitting in further fluid communication with the quick-release valve for venting the pressurized gas and terminating the blast shock wave to support a short and fast movement of the liquid media solution through the microfluidic channel.

8. The microfluidic system of claim 7, wherein the connector fitting is a T-connector.

9. The microfluidic system of claim 1, wherein the microfluidic channel is generally hexagonal.

10. The microfluidic system of claim 9, wherein the microfluidic channel has a length of approximately 5 mm.

11. The microfluidic system of claim 9, wherein the microfluidic channel has a maximum width of approximately 5 mm.

12. The microfluidic system of claim 1, wherein the duration of the blast shock wave is in milliseconds.

13. The microfluidic system of claim 1, wherein the pneumatic device is in operative communication with a pressurized tank, wherein the pressurized tank comprises a valve and a plurality of 0-rings engaged to the valve such that the plurality of 0-rings restricts a travel distance of the valve.

14. The microfluidic system of claim 1, wherein the bottom structure comprises polydimethylsiloxane.

15. A method of assembling a microfluidic system for precisely controlling shear forces generated by a blast shock wave, the method comprising:
forming a microfluidic channel defined by a bottom structure with a cover slip forming the bottom surface of the microfluidic channel, the microfluidic channel including an inlet and an outlet;
screening a surface of the microfluidic channel to define a cell plating region;
coating the cell plating region with a cellular matrix;
engaging an inlet reservoir to the inlet of the microfluidic channel;
forming a baffle extending within the inlet reservoir, the baffle having a baffle opening defining a restricted aperture, wherein the baffle is in fluid flow communication with the inlet of the microfluidic channel and supports laminar flow through the microfluidic channel;
engaging an outlet reservoir to the outlet of the microfluidic channel; and
filling the microfluidic channel, inlet reservoir and outlet reservoir with a liquid media solution.

16. The method of claim 15, wherein coating the cell plating region with a cellular matrix further comprises:
providing growth media to the cell plating region;
periodically replacing a portion of the cell plating region; and
labeling the cellular matrix.

17. The method of claim 15, further comprising marking a corner of the cell plating region on another surface of the cover slip, the other surface opposite the surface including the cell plating region.

18. The method of claim 15, further comprising marking a corner of the cell plating region on another surface of the cover slip, the other surface opposite the surface including the cell plating region.

19. The method of claim 15, wherein the cellular matrix comprises dissociated cells of a human central nervous system.

20. The method of claim 16, further comprising:
connecting one portion of a connector fitting to the inlet reservoir;
connecting a pneumatic device to another portion of the connector fitting; and
connecting a quick-release valve to another portion of the connector fitting for releasing a pressurized gas within the connector fitting and terminating a blast shock wave initiated by the pneumatic device.
